(19) United States
(12) Patent Application Publication
(10) Pub. No.: US 2010/0189793 A1
(43) Pub. Date: Jul. 29, 2010

(54) ELECTROPHORETIC TRANSDERMAL DELIVERY SYSTEM

(75) Inventors: Elisabeth MEYER, Miesbach (DE); Britta SCHRÕDER, Unterhaching (DE); Christine ZOGLMEIER, Grosskarolinenfeld (DE); Ulrich NICKEL, Erlangen (DE)

Correspondence Address: THE MAXHAM FIRM, 9330 SCRANTON ROAD, SUITE 350, SAN DIEGO, CA, 92121, US

(73) Assignees: ACINO AG, MIESBACH (DE); FRIEDRICH-ALEXANDER-UNIVERSITAT ERLANGEN-NUMBERG, ERLANGEN (DE)

(21) Appl. No.: 12/646,780

(22) Filed: Dec. 23, 2009

Related U.S. Application Data
(63) Continuation of application No. PCT/DE2008/001070, filed on Jun. 25, 2008.

(30) Foreign Application Priority Data
Jun. 25, 2007 (DE) 10 2007 029 139.8

(51) Int. Cl. A61K 9/14; A61K 31/445; A61K 31/4355; A61K 31/407; A61K 31/27; A61K 31/439; A61K 31/422; A61K 31/4045; A61P 25/04; A61N 1/30

(52) U.S. Cl. 424/484; 514/329; 514/279; 514/411; 514/490; 514/299; 514/376; 514/415; 604/20

(57) ABSTRACT

A transdermal delivery system (TDS) for administering at least one active agent through the skin. The system has a first electrode, a second electrode, and a matrix located between the first and second electrodes. The TDS contains at least one active agent which is either alkaline or acidic. The system is configured such that when applying a voltage to the electrodes, the pH of the matrix, in the case of an alkaline active agent, does not exceed the pH of the active agent and, in case of an acidic active agent, does not fall below the pH of the active agent.

### FIELD OF THE INVENTION

The present invention relates to an electrophoretic transdermal delivery system (TDS) for the administration of at least one active agent through the skin.

### DISCUSSION OF PRIOR ART

Transdermal therapeutic systems are used to deliver active agents through the skin into the blood vessel system. The stratum corneum of the skin, a natural barrier, must be overcome by the active agents. Passive diffusion, which is a result of the concentration gradient of the active agent between the TDS and the stratum corneum, is utilized to transport active agents through the skin.

Skin penetrating active agents usually enter the blood stream without further modification. However, the release of the agent from the TDS can only be controlled to a certain extent. The release of the active agent results in a relatively fast decrease of the agent's concentration in the TDS layer facing the skin so that the permeation rate through the skin tends to vary. Furthermore, such a passive concentration-dependent diffusion does not allow driving bigger or more complex agent molecules through the skin, like hormones, for example.

The permeability of the stratum corneum to an active agent is the most important limiting factor for a transdermal therapeutic system. A sufficient amount of the active agent must pass through the skin and into the blood stream in order to have the desired therapeutic effect. Improved skin permeation can be achieved by means of iontophoresis, which enables a pulsatile transport of the active agents through the skin. Iontophoresis involves the transport of ionized molecules along an electrical field that penetrates the skin. The electric field is typically generated by two electrodes that are affixed to the skin and supplied by a power source. The active agent molecules, which are available in ionized form, are extracted from the electrically conductive active agent reservoir and injected into the skin along the electrical field formed by the electrodes. One of the benefits of iontophoresis is the possibility of making macromolecules, even peptides like insulin, partially available through the skin.

In principle, iontophoretic systems are configured with a cathode and an anode to generate a direct current flow through the body. Aqueous buffer solutions, sometimes immobilized in gels, connect the electrodes for guaranteeing the flux of active agent ions through the skin. The electrodes have to be fixed on the skin with sufficient clearance between the cathode and the anode to avoid short-circuiting them. For this reason, iontophoretic systems are large in size. Iontophoretic systems have been described in a number of documents, including U.S. Pat. No. 3,991,755, U.S. Pat. No. 4,141,359, DE 37 03 321, WO 87/04936, WO 91/16077, WO 92/04938, EP 0 532 451, U.S. Pat. No. 5,415,629, and WO 00/53256.

Electrophoretic transdermal systems enable an injection of ionic active agents through the skin by using two electrodes and a power source, but without directly exposing the body to current flow. The electrodes of these systems are arranged face-to-face, similar to a capacitor, and the active agent reservoir is located between the electrodes with only one of the electrodes resting against the skin.

An example of such an electrophoretic TDS is described in U.S. Pat. No. 5,533,995. It is comprised of a reservoir and an electrode system adapted to deliver the active agent in a controlled manner from the reservoir into the skin.

A contact adhesive TDS having an electrode array is described in EP 1 457 233. This system is comprised of a means for electronic control of the electrode voltage, a supporting film (backing layer) that is impermeable to the active agent, a membrane permeable to the active agent, a reservoir containing the active agent which is located between the supporting film and the membrane, and a release liner. The electrode fixed to the supporting film serves as a counter electrode to the electrode provided at the membrane. The system further comprises a pressure-sensitive contact adhesive to attach the TDS to a skin surface.

### SUMMARY OF EMBODIMENTS OF THE INVENTION

It is a purpose of the present invention to provide an improved electrophoretic TDS compared to the prior art.

Embodiments of the present invention relate to a transdermal delivery system (TDS) comprising a matrix containing at least one alkaline or at least one acidic active agent, a first electrode, and a second electrode configured to be penetrated by the at least one active agent. The matrix of the delivery system is located between the first electrode and the second electrode. Where the TDS comprises an alkaline active agent, the matrix has a pH smaller than the pH of the alkaline active agent. Similarly, where the TDS comprises an acidic active agent, the matrix has a pH which is higher than the pH of the acidic active agent. The system is configured such that when a voltage is applied to the electrodes, the pH of the matrix does not exceed the pH of the active agent when that active agent is alkaline and does not fall below the pH of the active agent when that active agent is acidic.

The present invention further relates to a method for administering an active agent through the skin, wherein the TDS is attached to a region of the skin on the body of a human or an animal and left there for a predetermined delivery time period. During this predetermined time period a voltage is applied one or more times between the first and the second electrodes to administer the active agent such that the pH of the matrix does not exceed the pH of the active agent when that active agent is alkaline and does not fall below the pH of the active agent when that active agent is acidic.

The present invention further comprises the use of a TDS as defined above for administering at least one active agent through the skin.

The TDS according to the invention enables the release of one or more active agents in quantities that allow the provision of predetermined plasma levels to the patient over extended periods of time, such as 24 hours or, in particular, 72 hours. It should be understood that these time periods are examples only and any practicable elapsed times could be employed.

All acidic or alkaline active agents can be used, including those that feature a systemic effect and are suited for skin peilneation. Since the active agent according to the invention is not entirely neutral, but is in ionic form to at least a considerable degree, the risks of oxidation or precipitation, or both, of its neutral form having low water-solubility is small.

According to embodiments of the invention it is also possible to minimize the side effects of the active agent administered because the active agent can be released as needed for a limited time, thus restricting the active agent's release into the blood stream to only when required.

In an advantageous embodiment of the invention, at least 75%, and in particular at least 90%, and preferably at least 99%, of the alkaline active agent of the TDS is provided in protonated form (acidic active agents analogously in deprotonated form). Such an embodiment guarantees that a predominant portion of the active agent is available in a readily soluble form, which results in a TDS with a long shelf life. This embodiment further simplifies the pH control.

The matrix contains at least one active agent that is either protonatable or deprotonatable, that is, an acidic or alkaline active agent, which can include alkaline compounds or alkaline salts, acidic compounds or acidic salts, and where appropriate, zwitterionic compounds.

Particularly suited for the widest range of applications of the TDS according to embodiments of the invention are alkaline or acidic active agents selected from analgesics, μ-opioid-receptor-antagonists, anaesthetics, parasympathomimetics, parasympatholytics, antiemetics, emetics, sympathomimetics, hormones, anti-migraine agents, antiallergics, anti-convulsants, anti-dementia drugs, antidepressants, beta receptor blocker, analeptics, and mixtures thereof.

Alkaline active agents are preferably selected from fentanyl, buprenorphine, physostigmine, rivastigmine, scopolamine, granisetron, zolmitriptan, sumatriptan, salbutamol, and mixtures thereof.

According to a preferred embodiment of the invention, the matrix contains one or more additives to improve the flux of the active agent through the skin. Such additives are selected from penetration-enhancing compounds or permeation-enhancing compounds, or both, preservatives, antioxidants, humectants, electrolytes, thickening agents, emulsifiers, tackifiers, plasticizers, acidifying agents, alkalifying agents, buffers, fillers, and mixtures thereof.

A favorable reduction of gas formation within embodiments of the invention is achieved when at least the electrode being used as the anode is made of silver and when the matrix contains chloride (Cl⁻).

Embodiments of the TDS according to the invention further comprise a pressure-sensitive adhesive layer configured to contact the skin upon application of the system, thus enabling a straightforward attachment to the skin. Delays in the delivery of active agents to the skin can be minimized when the adhesive layer also contains the at least one alkaline or the at least one acidic active agent in a properly selected concentration.

In an embodiment of the invention, a backing foil impermeable to the active agent, and preferably permeable to gas, is provided at the side of the TDS facing away from the skin. This prevents any accidental release of the active agent from the system and simultaneously allows the system to discharge gases generated by electrolysis.

For an energy-self-sufficient use of the system, the TDS can further include a voltage supply, which preferably comprises a voltage source and a control device. A high degree of operational reliability can be achieved with one or more spacers or one or more support elements, or both, whereby the fixation of these elements in the system can be improved with one or more pressure-sensitive contact adhesive layers. The TDS can further comprise a removable release liner serving as protection against contamination and accidental release of active agents.

### BRIEF DESCRIPTION OF THE DRAWING

The invention is further described below by means of the subsequent detailed description of advantageous embodiment examples of the invention, reference being made to accompanying drawing, wherein:

FIG. 1 is a schematic representation of a cross section through an electrophoretic TDS according to the invention;

FIG. 2 is a chart illustrating the flux of an active agent through the skin for different TDSs;

FIG. 3 is a chart illustrating the accumulated release of the active agent through the skin for the TDSs shown in FIG. 2,

FIG. 4 is a chart illustrating the flux of the active agent through the skin for different voltages applied to electrodes of an electrophoretic TDS according to the invention over different periods of time; and

FIG. 5 is a chart illustrating the development of fentanyl blood levels over time, when the fentanyl has been administered using an electrophoretic TDS according to embodiments of the invention with two voltage cycles applied.

### DETAILED DESCRIPTION OF EMBODIMENTS OF THE INVENTION

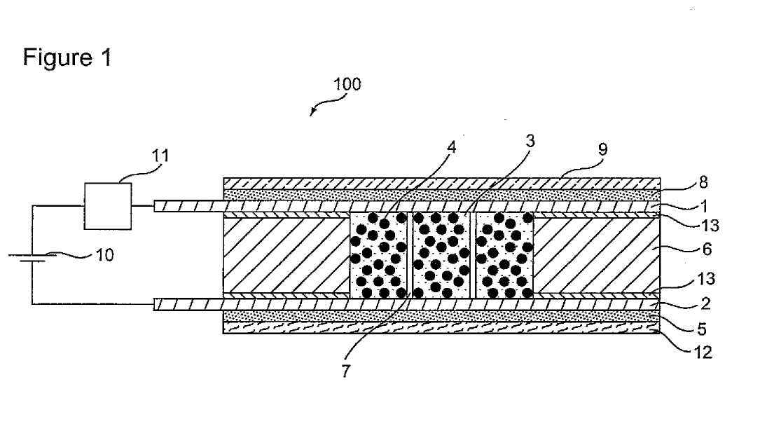

The TDS 100 illustrated in FIG. 1 comprises two electrodes, electrode 1 and electrode 2, with matrix 3 arranged between the electrodes. Matrix 3 contains active agent 4. Adhesive layer 5 is provided at the side of lower electrode 2 which faces away from matrix 3, for attaching the TDS to a surface of a skin. The surface of adhesive layer 5 facing away from the electrode is referred to herein as the skin-facing surface of TDS 100.

Lower electrode 2 is formed with openings allowing the active agent to pass through to adhesive layer 5. Upper electrode 1 can comprise openings too, but may also be implemented with a closed surface. The side faces of matrix 3 located between the electrodes are enclosed by spacer 6 which prevents the active agent or the matrix from flowing out of the system and which guarantees that electrode 1 and electrode 2 will not come into contact with each other. If matrix 3 is to span a bigger area, the electrode gap within the matrix area is conveniently maintained using one or more support elements 7. The side of electrode 1 facing away from matrix 3 has adhesive layer 8 thereon for attaching backing foil 9. The side of adhesive layer 5 that faces the skin can be covered with a removable release liner 12 for protecting it against contamination when not in use.

Electrode 1 and electrode 2 are each configured to be connected to a voltage supply. As indicated in FIG. 1, the voltage supply advantageously comprises voltage source 10, implemented in the form of a battery, for example, and device 11 for controlling the level and temporal course of the voltage applied to the two electrodes. The voltage supply can be integrated into TDS 100, whereby the control is preferably implemented as a microelectronic unit, in the form of an integrated circuit, for example. The electrodes as well as the voltage supply can alternatively or additionally also be configured to be connected to an external voltage supply.

The configuration of TDS 100 explained above serves to transport one or more active agents 4 contained in matrix 3 in a controlled manner for an extended period of time and in an adequate concentration through skin facing electrode 2, subsequent adhesive layer 5 and onto the skin surface on which the system is attached by means of adhesive layer 5.

It is principally possible to provide the matrix in liquid form, in gel form, or in the form of a self-supporting solid. It is appreciated that a person skilled in the art will, in case of a liquid or flowable matrix, make provisions against a “leakage” of the matrix or of the active agent or agents contained therein. Examples include the provision of a solid envelope that is impermeable to liquids and that borders at the skin facing side to a membrane being permeable to active agents, or the thickening of the matrix by using suitable gelling agents. The matrix is preferably polymer-based.

Basically all polymers used for the fabrication of transdermal systems that are physiologically harmless are suited for fabricating the matrix, provided that the ploymers are hydrophilic and, if necessary, amphiphilic, and either contain water or can absorb water. Particularly suited are hydrogels such as synthetic hydrogels like poly(meth)acrylic acids, poly(meth)acrylates, polyvinylpyrrolidone, or polyvinyl alcohol. Cellulose-based hydrogels are also suited, like cellulose ethers, for example. Hydroxyalkylcelluloses, such as hydroxymethylcellulose, hydroxyethylcellulose, or hydroxypropylcellulose are used in particular. The matrix can be tacky where required, but alternatively an adhesive layer (not shown in FIG. 1) can be provided between the matrix 3 and the adjacent electrodes.

The term “matrix” as used in this specification is to be understood such that the matrix either already contains water and is as such suitable for storage, or absorbs water either upon inserting water just before an application of system 100 or, where appropriate, from the skin.

Matrix 3 contains one or more active agents 4 for which either alkaline or acidic active agents can be used. Such active agents are characterized by their convertibility into an ionic form. A TDS using an alkaline active agent is described below. It should be noted that the functionality described applies analogously also to acidic active agents. The active agents may be contained in the matrix in the form of their pharmaceutically acceptable salts.

The pH in the matrix of system 100 according to the invention is adjusted to a value smaller than the pH of the active agent, meaning to a value where at least 50% of the alkaline active agent exists in protonated form as cation. In preferred embodiments, at least 75%, preferably at least 90%, particularly preferably 99%, and advantageously at least 99.9% of the alkaline active agent is provided in protonated form. These high concentrations of the protonated active agent guarantee that the active agent exists in dissolved form, because the neutral form of the active agent has a low water-solubility and can precipitate from the solution when a certain concentration is exceeded resulting in an immobilization of the active agent and rendering it no longer freely available.

Examples of suitable active agents are analgesics, μ-opioid-receptor-antagonists, anaesthetics, parasympathomimetics, parasympatholytics, antiemetics, emetics, sympathomimetics, hormones, anti-migraine agents, antiallergics, anticonvulsants, anti-dementia drugs, antidepressants, beta receptor blocker, and analeptics.

The analgesics can involve opioids, of which full agonists, agonists-antagonists, partial antagonists, and full antagonists can be quoted. Full agonists are for instance fentanyl, remifentanil, oxycodone, and methadone. From the full antagonists, naloxone and naltrexone can be mentioned. An example of an agonist-antagonist is nalbuphine, and a partial antagonist is, for instance, buprenorphine. Salicylic acid derivatives like acetylsalicylic acid, etofenamate, or diclofenac are examples for acidic analgetics.

Of the μ-opioid-receptor-antagonists, almivopan and methylnaltrexone can be mentioned.

Of the anaesthetics, local anaesthetics such as lidocaine, tetracaine, or etidocaine are to be considered.

Cholinesterase inhibitors are examples for parasympathomimetics, whereby physostigmine, rivastigmine, neostigmine, donepezil, and galantamine are to be mentioned in particular.

Scopolamine can, for example, be quoted for the parasympatholytics.

The antiemetics can be selected from the parasympatholytics, 5-HT₃ -receptor-antagonists like ondansetrone and granisetron, and dopamine-D₂-receptor-antagonists like domperidone.

Of the emetics, dopamine-D₂-receptor-agonists like apomorphine are to be considered.

From the sympathomimetics chatecholamines like dobutamine have to be considered. Further, β₂-sympathomimetics are to be mentioned, like salbutamol, fenoterol, and clenbuterol.

The hormones can be selected from estradiol, norelgestromin, goserelin, or buserelin.

Examples for anti-migraine agents are 5-HT₁-receptor-agonists like triptans and, of these, zolmitriptan, sumatriptan or naratriptan in particular.

An example for anticonvulsants is gabapentin.

Memantine can be mentioned as anti-dementia drug.

Of the antihistamines, antiallergics are to be considered, like mizolastine, triprolidine, and desloratadine, for example.

Of the antidepressants, nortriptyline can be quoted.

An example of a suitable beta-blocker is nebivolol.

Methylphenidate is an example for an analeptic.

The matrix may also contain more than one active agent, for instance, a combination of two active agents, such as a parasympathomimetic in combination with a parasympatholytic, in particular physostigmine/scopolamine, an analgetic in combination with an antiemetic, like fentanyl in combination with granisetron, or two analgetics, like a μ-receptor-agonist and μ-receptor-antagonist, for example, fentanyl/naloxone.

For the transport through the skin and optional adhesive layer 5, active agent 4 has to be available in deprotonated, that is, neutral form. The conversion of a portion of protonated active agent 4 contained in the matrix is achieved by applying a voltage to the electrodes. This generates not only an electric field penetrating matrix 3 that gives rise to a migration of the ions within the matrix, but causes also electrochemical processes at the electrode surface.

Particularly the electrons injected from the cathode into the system react with the hydronium ions (H₃O⁺) to form molecular hydrogen, whereby hydroxide ions (OH⁻) remain in the immediate vicinity of the cathode. The hydroxide ions react with the protons of the protonated active agent, thereby deprotonating the active agent and converting it into its neutral form. The locally increased OH⁻-concentration causes an increased pH. For preventing a crystallization of the deprotonated active agent, which would prevent its migration to the skin surface, the pH should not exceed the pH of active agent 4 at any location within matrix 3.

The hydroxide ions donate electrons to the anode thereby forming molecular oxygen and leaving hydronium ions at the electrode. The gas bubbles developed by this process can affect the controlled application of the TDS. As a preventive measure, electrode 1 serving as anode and covering sheet 9 can be configured to be permeable to gases. For covering sheet 9, foils can be used which are known to the person skilled in the subject matter of the art and made for instance of polyesters, polyolefins like polyethylenes or polypropylenes, polyvinylidene chloride or polyurethanes with a thickness of 5 μm to 1000 μm. Perforated, porous, or fabric-like foil materials are, inter alia, suited for gas-permeable electrodes.

The development of gases can further be minimized by using a silver anode together with an adequate amount of chloride in the matrix. Only a small amount of oxygen, if any, is formed in this case at the anode because silver is, for the most part, oxidized according to the reaction Ag+Cl⁻→AgCl+e⁻, thereby forming silver chloride that is deposited at the surface of the anode. The term “silver anode” as used in the present context refers to an anode which is either formed of silver or which comprises a silver plating at its surface facing the matrix. Silver plated stainless steel fabrics and also perforated silver foils or silver plated polyester fabrics are preferred for the silver anodes.

Due to the reduced generation of hydronium ions in the anode region when using silver anodes, the pH also rises in the vicinity of the anodes and thus enables the deprotonation of the active agent within the entire matrix region. Since this embodiment is different from the representation of FIG. 1, it is therefore possible, in this particular case, to run the anode as a skin-facing electrode. A further advantage of this would be that the hydrogen formed at the cathode could degas through the cathode and the covering sheet without having to drift through the matrix.

The cathode and the anode are preferably formed by gas-permeable silver electrodes, that is, electrodes made of silver or silver plated material. Principally all conventional noble metals can be used as electrodes, for example, electrodes either made of gold, platinum, or palladium or plated therewith, but also stainless steel electrodes and copper or copper-plated electrodes. Other suitable electrodes are those made on a carbon basis. The anode can, of course, also be made from other materials than those used for the cathode.

The electrodes can again be made in the form of a fabric, preferably a grid-like fabric, in the form of perforated or porous foils, or in the form of foil printed or pattern printed with an electrically conductive material. Particularly upper electrode 1 can be formed by an electrically conductive pattern printed on the covering sheet. The mesh size when using a grid-like electrode or a grid-like printed electrode can be 0.001 mm to 1 mm, for example, and in particular 0.01 mm to 0.05 mm.

The adhesion of TDS 100 on a skin surface can be improved by using an adhesive layer. Adhesive layer 5 is, of course, adapted for enabling active agent 4 to diffuse through it.

Potential contact adhesives for adhesive layer 5 are known to the person skilled in the art, and for this purpose, homopolymers and copolymers on an acrylic basis which preferably have no functional groups, natural and synthetic rubbers, polyvinyl acetate, polyisobutylene, and silicone based adhesives are to be mentioned in particular. Preferably a hydrophobic contact adhesive is used. The TDS can alternatively also be provided with an additional adhesive tape for being fixed to the skin, which is usually also referred to as overtape.

In its original condition, that is, before any voltage is applied to the electrodes, adhesive layer 5 can be free of active agents. In this case, the active agent has to penetrate the adhesive layer after a start-up of system 100 before it can reach the skin surface. For preventing a respective delay in the delivery of the active agent to the skin, adhesive layer 5 can be provided with an appropriate initial active agent concentration, which guarantees an immediate, or at least an early, start of the permeation of the active agent through the skin. The area weight of adhesive layer 5 can for instance be in the range from 10 g/m2 to 100 g/m2.

The concentration of active agent or active agents 4 in matrix 3 or in adhesive layer 5, or both, depends on the application time of the TDS, the favoured release rate, and the permeation of the active agent through the skin. It can vary within a broad range. Typical concentrations of active agent 4 range from about 0.1% by weight to about 10% by weight based on the total weight of matrix 3.

Matrix 3, as well as adhesive layer 5, can contain additives typical for TDSs like, for instance, penetration enhancing or permeation enhancing compounds, or both, preservatives, antioxidants, humectants, electrolytes, thickening agents, emulsifiers, tackifiers, plasticizers, acidifying agents, alkalifying agents, fillers, and mixtures thereof. It is appreciated that a person skilled in the art will select one or, where required, more additive(s) and their quantity proportions such that the beneficial properties of the electrophoretic TDS are not affected or not substantially affected.

Removable release liner 12 protects the transdermal system against contamination and untimely release of the active agent in the usual manner. Siliconized polyethylene terephthalate foils or fluoro-polymer coated polyester foils, for example, can be used.

Spacer or spacers 6 are preferably produced from a foam-like material. When using non-tacky materials, the surfaces of spacers 6 contacting the electrodes can be provided with adhesive layer 13. Normally polytetrafluoroethylene (sold under the trademark Teflon) rings can be used for support element or support elements 7.

Delivery system 100, explained with reference to FIG. 1, enables an adjustment of the pH by electrolysis to a value which is high enough, at least in the cathode region, for releasing a sufficient amount of uncharged active agent that can give rise to its subsequent diffusion through the matrix and adhesive layer 5, if present, and through the skin. However, the pH should not so high that the uncharged active agent crystallizes and can no longer be delivered due to the immobilization associated therewith.

An advantage of the system is that the effective concentration of the active agent in the blood can be achieved on demand and in a short amount of time. It can also be increased very fast if needed, whereby “fast” means periods of time of less than 120 min., preferably less than 60 min. and, in particular, less than 30 min. This allows an easy adjustment of the blood level of the active agent to changing needs.

The flux of the active agent through the skin can be well controlled by the amount of the active agent converted into its neutral form, thereby enabling keeping the desired concentration of the active agent in the blood over long periods of time like two, three, or more days, for example.

Furthermore, a more effective use of the amount of active agent contained in a TDS is achieved because compared with conventional systems more of the active agent can be released from the system. This represents an additional advantage both with regard to the required amount of active agents which are, for the most part, very expensive, and with regard to the possibly required disposal of the used delivery systems.

The specific release of the active agent using an appropriate control of the electrode voltage enables an accurate control of the permeation of the active agent, and thus, an adjustment of the blood level of the active agent within a therapeutic window and over long periods of time.

The initial pH of the system, that is, the pH before a voltage is initially applied to electrodes 1 and 2, can, for instance, be adjusted to a desired value below the pH of the active agent used by acidifying the matrix with a physiologically acceptable inorganic or organic acid or mixtures thereof. Hydrochloric acid, phosphoric acid, citric acid, sorbic acid, and acetic acid are examples of suitable acids.

According to a preferred embodiment, matrix 3 can further contain a conventional buffer since this enables a better control of the pH increase. The buffer can be an acetic acid/acetate buffer or a citric acid/citrate buffer, for example.

When using an active agent with a pH around 9, examples being fentanyl (pH=8.4) or granisetron (pH=9.4), the initial pH is preferably located in the range from 2 to 7, more preferably in the range from 3 to 6, and most preferably around 4.

When applying a voltage to the two electrodes, the pH increases at the cathode as explained above and enables a deprotonation of the active agent.

The voltage has to be sufficiently high for the water electrolysis to take place. The threshold value for the voltage applied depends on both the electrode material used and the electrolyte used in the matrix. A person skilled in the art can determine the appropriate voltage value in each case based on the developing gas formation, the measurable current flow or using a suitably selected pH-indicator, among others. It has been found that voltages from about 2 V and higher are well suited to most of the systems.

The amount of charge supplied must not be allowed to be too high so that the pH does not exceed the pH of the active agent used. This means that the current supplied or the duration of the current supplied, or both, have to be limited correspondingly.

According to a particularly preferred embodiment of the invention, a controlled increase of the pH in the cathode region is achieved by repeatedly applying a specified voltage for a predetermined period of time. The voltage used, the time period during which the voltage is applied, and the repetition intervals depend again on the specific delivery system used and, in particular, on the active agent contained therein. Typical ranges are a voltage of 2 V to 6 V for a period of 10 sec. to 60 sec. with a repetition rate of 12 hr. to 24 hr. It is appreciated that also shorter periods can be chosen, during which the electrode voltage is applied, whereby the application of the electrode voltage will then usually have to be repeated in shorter intervals. The electrode voltage may, for instance, be applied for periods of 2 sec. to 10 sec., which are repeated in intervals of 1 to 15 hours. The permeation of the active agent can be adjusted to the desired therapeutic window by the ratio between the time period and the repetition rate.

The variation of the pH can be accurately controlled within a time period when using a modulated and, in particular, when using a pulse-width modulated electrode voltage since the modulation allows to control the flux of electrons between electrodes and matrix to a great extent independent of the electrode voltage. This modulation can be achieved by means of the duty cycle, for example. The modulation is preferably applied with frequencies ranging from a few Hertz to some kHz.

The pH can be screened or monitored, or both, using a pH-indicator inserted into the matrix, whereby the transition point of the indicator is preferably harmonized with the desired pH below the pH of the active agent. All pH-indicators which are toxicologically harmless and which do not affect the favorable properties of the delivery system can be used,. Examples of suitable pH-indicators are phenolphthalein, bromothymol blue, phenol red, and the like.

Below an example is given for manufacturing an embodiment of TDS 100 as described above.

Pressure-sensitive adhesive layer 13 is applied to both faces of a physically cross-linked closed-cell polyolefin foam of approximately 1 mm thickness. A ring serving as spacer 6 for the electrodes and having an outer diameter of about 20 mm and an inner opening of about 1 cm² is punched out of the foam.

A hydrogel inserted into the opening serves as matrix 3. The hydrogel is formed of water and about 2.5% by weight hydroxyethylcellulose. Fentanyl citrate is introduced into the hydrogel as active agent 4. The pH of matrix 3 is adjusted to a value of about 4. The electrodes are provided on both sides of matrix 3, each having a diameter of about 16 mm. A silver-plated stainless steel square-mesh fabric with a mesh size of about 0.06 mm is used for the anode (electrode 1) as well as for the cathode (electrode 2). Each of the electrodes comprises a strip-like lead adapted for connection to external voltage supply and control 10, 11. Transparent covering foil 9 coated on one side with a self-adhesive contact adhesive is applied to the side facing away from the skin. Release liner 12 coated with a pressure-sensitive hydrophobic silicone adhesive is applied to the lower or skin side facing away from covering foil 9. Release liner 12 has, of course, to be removed before applying the TDS to the skin.

It is understood that the indicated shapes, dimensions, active agents, additives, and specifications of concentrations represent only specific examples for TDS 100 according the invention. Depending on the type of applications, each TDS according to the invention can, of course, be implemented with other shapes, dimensions, active agents, additives, or concentrations, which a person skilled in the art is capable of customizing easily.

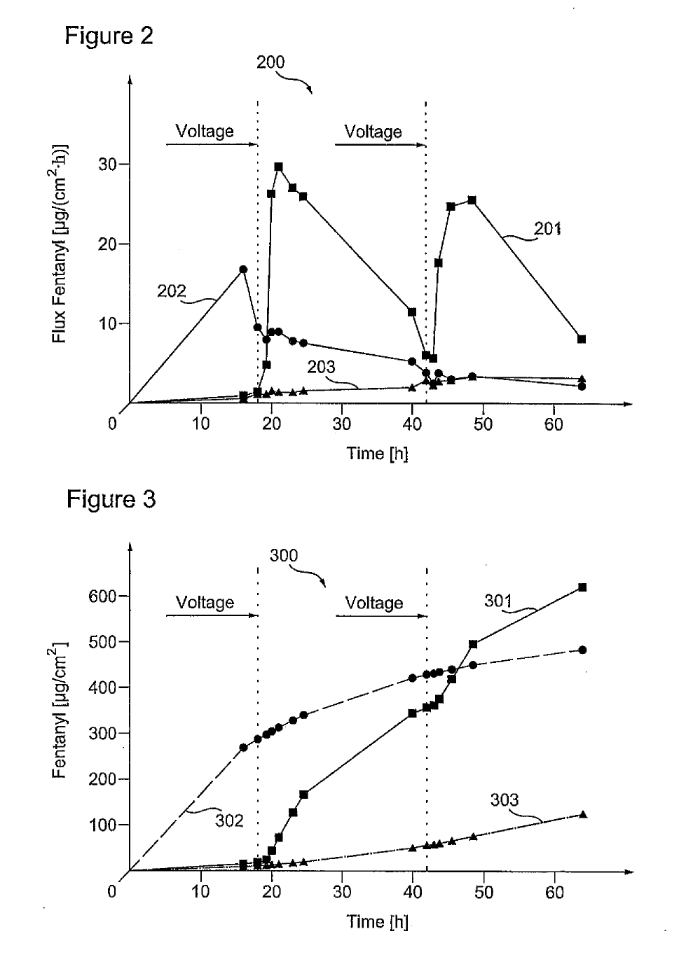

The flux of active agent versus time measured in an in vitro skin permeation test for different transdermal delivery systems is shown in the diagram of FIG. 2. The values shown represent average values obtained from n measurements. Graph 201 (n=3) represents the permeated flux (permeation rate) of the active agent fentanyl out of an electrophoretic TDS as described above, whereby a voltage of 2 V has been applied to the electrodes of the TDS a first time 18.5 hours after the commencement of the test and for 60 seconds. A voltage of 3 V has been applied again 42.5 hours after the commencement of the test for an equal length of time. Graph 202 (n=3) shows the permeation flux achieved with fentanyl-containing transdermal matrix system “Durogesic® SMAT 25 μg/h” available on the market from the company, Janssen-Cilag. The release of the active agent is effected with this system only passively, that is, without applying an electrode voltage. Graph 203 (n=2) represents the flux of the active agent achieved with a TDS according to the invention without applying an electrode voltage.

The release of the active agent by the passive Durogesic matrix system increases for the first 16 hours of the test, followed by, in a first approximation, an exponential decrease. Compared to the Durogesic matrix system, the release of active agent by electrophoretic TDS 100 increases only hesitantly and achieves no noteworthy flux even after 17 hours. However, when applying an electrode voltage (graph 201) at about 18 hours after commencing the test, the permeation rate increases within a short time period and finally arrives at a maximum value of nearly two-fold of that achieved with the Durogesic matrix system. The increase of the permeation rate spans a period of about 2 hours, which is much longer than the period during which a voltage has been applied to the electrodes. After having passed the maximum permeation rate, the release of active agent, in a first approximation, decreases linearly and significantly more slowly as compared to the Durogesic matrix. Only after more than 20 hours, the fentanyl flux corresponds approximately to that of Durogesic matrix, which is a sign of the depletion of the active agent provided in neutral form in the TDS. Applying again an electrode voltage of 3 V about 42 hours after commencement of the test results in a further increase of the active agent release within a short time period, whereby the thus achieved maximum permeation rate is now slightly lower than after the first voltage interval. An approximately linear decrease of the active agent release is observed following its maximum value.

Different from a voltage-controlled operation of electrophoretic system 100, the permeation rate increases only slowly in the absence of a voltage control (graph 203) and only to comparably low values (about 10 percent of the maximum value achieved after application of an electrode voltage).

With a voltage controlled electrophoretic system, higher permeation rates can be achieved for a longer period of time than with commercially available passive systems (see graph 202), whereby the desired permeation rate can be adjusted with parameters like electrode voltage, duration of an electrode voltage application, and repetition rate of the electrode voltage application. It follows that these parameters allow the level of the active agent in the blood of a user of the TDS to be kept within a required range and this level of the active agent is obtained much faster and can be maintained for a comparatively longer period of time than with passive systems.

In addition, the total amount of active agent delivered to the skin per unit area with electrophoretic TDS 100 is considerably larger than with passive systems. This is evident from diagram 300 of FIG. 3, which shows a plot of the amounts of active agents released by the systems shown in FIG. 2 over time. The plot is a cumulative representation wherein each point of the graph indicates the amount of active agent released by the respective TDS up to that time.

While the accumulated amount of active agent released by the passive Durogesic system (graph 302) levels off increasingly after the maximum permeation rate has been passed and amounts to less than 500 μg/cm2 in total after 65 hours, the amount of active agent released by electrophoretic system 100 (graph 301) rises sharply shortly after applying the electrode voltage and adds up to a total of about 600 μg/cm2 in only about 40 hours after the first application of an electrode voltage. Without applying a voltage (graph 303), the amount of active agent released by electrophoretic system 100 increases only marginally, and reaches only a total amount of about 100 μg/cm², even after 65 hours.

The results presented explain that a fast delivery of an active agent is possible at high permeation rates and for a long period of time when using electrophoretic TDS 100 by repeatedly applying an electrode voltage for short time intervals.

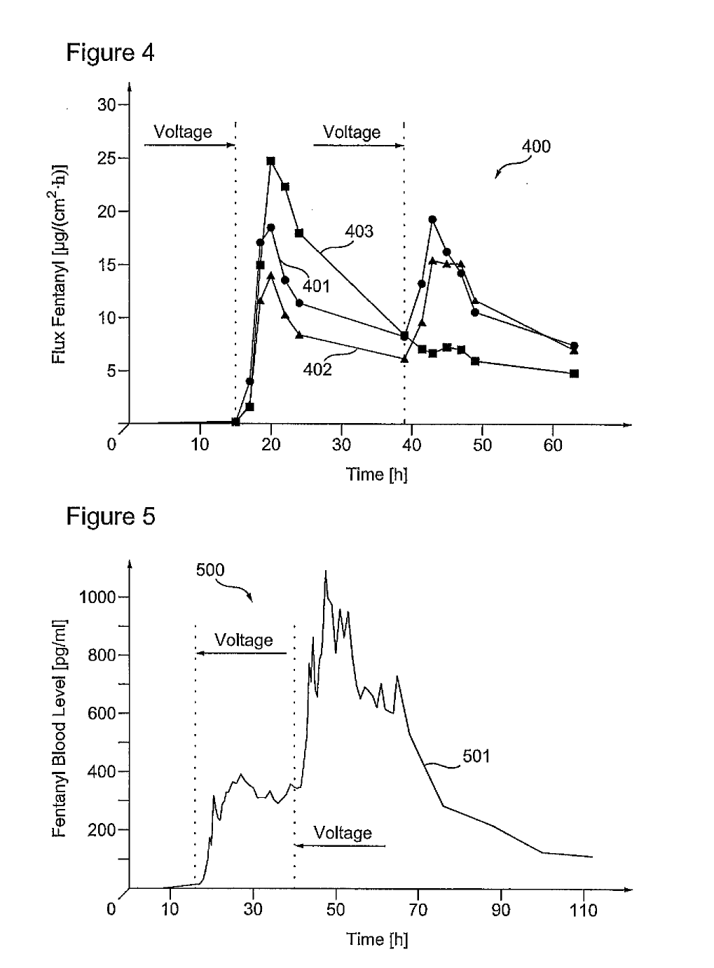

Diagram 400 of FIG. 4 shows the permeation flux of the active agent fentanyl effected with three identical samples of electrophoretic TDS 100, whereby the electrode voltages applied to the respective samples differ with respect to level and duration. The values shown are average values of n measurements. An electrode voltage has been applied to all three samples at the same time twice after commencement of the test. Before the electrode voltage has been applied for the first time, the permeation flux is very low for all three samples. It does not increase until the first application of the electrode voltage and approximately equally fast for all three samples. However, the maximum permeation flux achieved with the various samples varies significantly.

When applying an electrode voltage of 2 V for a period of 60 seconds (graph 401, n=2), the maximum permeation rate achieved amounts to about 19 μg/(cm²•h). When applying an electrode voltage of 3 V for 15 seconds (graph 402, n=3), the maximum permeation rate is less than 15 μg/(cm²•h). The highest maximum flux of about 25 μg/(cm²•h) is achieved with an electrode voltage of 9 V for 60 seconds (graph 403, n=3). The flux decreases thereafter, however, significantly faster than for the other two samples.

A voltage is applied to the electrodes for a further time about 20 hours after the first application of an electrode voltage. While the maximum permeation flux is, to a first approximation, reproduced for the first sample (graph 401) and for the second sample (graph 402), no significant increase of the permeation flux is observed for the third sample (graph 403). This can be explained by a crystallization of the deprotonated active agent, that is, by the active agent being rendered immobile.

Diagram 500 of FIG. 5 shows the results of a clinical test, where an electrophoretic TDS, as described above, had been administered to a test person. Graph 501 represents a plot of the fentanyl blood level of the test person versus the application time. A DC-voltage of 2.5 V has been applied to the electrodes for 60 seconds after the first 16 hours and then again 40 hours after affixing the electrophoretic system. The fentanyl blood levels of the test person have been monitored at regular intervals of one hour and immediately after applying the voltage in half-hour intervals. After 65 hours the blood level values have been recorded at longer time intervals.

It is evident from the diagram that the active agent has entered the blood stream after penetrating the initially no active agent containing adhesive layer 5 and the skin already within about 2 hours after application of a voltage. The blood level of the active agent increases rapidly and remains then on a basically constant level. By applying a voltage for a further time, the fentanyl concentration increases again within a short time period, maintains a high level for about 15 hours, and then decreases slowly. The system has been removed after 64 hours, whereby about three days after the first application of an electrode voltage, the fentanyl blood level is still above 200 pg/ml.

It is evident from this test that an electrophoretic TDS according to the present invention not only allows one to achieve a desired blood level of an active agent within a short time, but also that the blood level of an active agent can be increased further if required.

While the present invention has been shown and described herein in what is believed to be the most practical and preferred embodiments, it is recognized that many alternatives, modifications, and variations will be apparent to those skilled in the art. Accordingly, the exemplary embodiments of the invention set forth herein are intended to be illustrative and not limiting in any way. Various changes may be made without departing from the spirit and scope of the present invention as defined in the following claims.

What is claimed is:

1. A transdermal delivery system comprising:
a matrix containing at least one alkaline active agent;
a first electrode; and
a second electrode adapted for being penetrated by said at least one active agent;
whereby said matrix is located between said first electrode and said second electrode, said matrix has a pH which is lower than the pH of said at least one active agent, and whereby the system is configured such that a voltage applied to said first and second electrodes does not result in the pH of said matrix exceeding the pH of said at least one active agent, and wherein at least 75% of said alkaline active agent is provided in protonated form.

2. The transdenual delivery system according to claim 1, wherein at least 90% of said alkaline active agent is provided in protonated form.

3. The transdermal delivery system according to claim 1, wherein at least 99% of said alkaline active agent is provided in protonated form.

4. The transdermal delivery system according to claim 1, wherein said at least one alkaline active agent is selected from the group consisting of analgesics, μ-opioid-receptor-antagonists, anaesthetics, parasympathomimetics, parasympatholytics, antiemetics, emetics, sympathomimetics, hormones, anti-migraine agents, antiallergics, anticonvulsants, anti-dementia drugs, antidepressants, beta receptor blocker, analeptics, and mixtures thereof.

5. The transdermal delivery system according to claim 4, wherein said at least one alkaline active agent is selected from the group consisting of fentanyl, buprenorphine, physostigmine, rivastigmine, scopolamine, granisetron, zolmitriptan, sumatriptan, salbutamol, and mixtures thereof.

6. The transdermal delivery system according to claim 1, wherein said matrix contains one or more additives selected from the group consisting of penetration enhancing or permeation enhancing compounds, or both, preservatives, antioxidants, humectants, electrolytes, thickening agents, emulsifiers, tackifiers, plasticizers, acidifying agents, alkalifying agents, buffers, fillers, and mixtures thereof.

7. The transdermal delivery system according to claim 1, wherein at least said first electrode is used as an anode and is a silver anode, and wherein said matrix contains chloride (Cl⁻).

8. The transdermal delivery system according to claim 1, and further comprising a contact adhesive layer configured for contacting the skin upon application of the system.

9. The transdermal delivery system according to claim 8, wherein said contact adhesive layer also contains said at least one alkaline active agent.

10. The transdermal delivery system according to claim 1, and further comprising a backing foil impermeable to said active agent and being selectively permeable to gas.

11. The transdermal delivery system according to claim 1, and further comprising a voltage supply comprising a voltage source and a control device, spacers means, support element means and, selectively, pressure-sensitive contact adhesive layer means and removable release liner means.

12. A transdermal delivery system comprising:
a matrix containing at least one alkaline active agent being selected from the group consisting of analgesics, μ-opioid-receptor-antagonists, anaesthetics, parasympathomimetics, parasympatholytics, antiemetics, emetics, sympathomimetics, hormones, anti-migraine agents, antiallergics, anticonvulsants, anti-dementia drugs, antidepressants, beta receptor blocker, analeptics, and mixtures thereof;
a first electrode;
a second electrode adapted for being penetrated by said at least one active agent; and
a backing foil being impermeable to said active agent;
whereby said matrix is located between said first electrode and said second electrode and has a pH which is lower than the pH of said at least one active agent, and whereby the system is configured such that a voltage applied to said first and second electrodes does not result in the pH of said matrix exceeding the pH of said at least one active agent, and wherein at least 75% of said alkaline active agent is provided in protonated form.

13. A transdermal delivery system (100) comprising:
a matrix (3) containing at least one alkaline active agent (4) being selected from the group consisting of analgesics, μ-opioid-receptor-antagonists, anaesthetics, parasympathomimetics, parasympatholytics, antiemetics, emetics, sympathomimetics, hormones, anti-migraine agents, antiallergics, anticonvulsants, anti-dementia drugs, antidepressants, beta receptor blocker, analeptics, and mixtures thereof;
a first electrode;
a second electrode adapted for being penetrated by said at least one active agent;
a backing foil being impermeable for said active agent; and
a voltage supply comprising a voltage source and a control device;
whereby said matrix is located between said first electrode and said second electrode and has a pH which is lower than the pH of said at least one active agent, and whereby the system is configured such that a voltage applied to said first and second electrodes does not result in the pH of said matrix exceeding the pH of said at least one active agent, and wherein at least 75% of said alkaline active agent is provided in protonated form.

14. A transdermal delivery system comprising:
a matrix containing at least one acidic active agent;
a first electrode; and
a second electrode adapted for being penetrated by said at least one active agent;
whereby said matrix is located between said first electrode and said second electrode and has a pH which is higher than the pH of said at least one active agent, and whereby the system is configured such that a voltage applied to said first and second electrodes does not result in the pH of said matrix falling below the pH of said at least one active agent.

15. A method for administering an active agent through the skin, wherein a transdermal delivery system according to claim 1 is attached to the skin of a human or animal body for a predetermined delivery time period, during which time a voltage is applied one or more times between the first and second electrode such that in case of an alkaline active agent the pH of the matrix does not exceed the pH of the at least one active agent, and such that in case of an acidic active agent the pH of the matrix does not fall below the pH of the at least one active agent.

16. A method for administering an active agent through the skin, wherein a transdermal delivery system according to claim 12 is attached to the skin of a human or animal body for a predetermined delivery time period, during which time a voltage is applied one or more times between the first and second electrode such that in case of an alkaline active agent the pH of the matrix does not exceed the pH of the at least one active agent, and such that in case of an acidic active agent the pH of the matrix does not fall below the pH of the at least one active agent.

17. The use of a transdermal delivery system according to claim 1 for administering at least one active agent through the skin.

18. The use of a transdermal delivery system according to claim 17 for administering at least one active agent through the skin.
